(19) United States
(12) Patent Application Publication
(10) Pub. No.: US 2016/0331963 A1
(43) Pub. Date: Nov. 17, 2016

(54) Skin and Muscle Stimulation Apparatus

(71) Applicant: Innovate Photonics Limited, Swansea (GB)

(72) Inventor: David Simon George, Swansea (GB)

(21) Appl. No.: 14/718,243

(22) Filed: May 21, 2015

(30) Foreign Application Priority Data
May 14, 2015 (GB) 1508219.1

(51) Int. Cl. A61N 1/36; A61N 5/06

(52) U.S. Cl. CPC A61N 1/36014; A61N 5/0622; A61N 2005/0652; A61N 2005/0645

(57) ABSTRACT

Apparatus for applying light to an area of skin and for simultaneously electrically stimulating that area of skin, the apparatus including at least one flexible pad adapted to be placed onto or over the area of skin to be treated, the or each pad including a flexible circuit board providing, in use, electrical power to an electric muscle stimulation (EMS) electrode and an array of Light Emitting Diodes (LEDs) interspersed therebetween whereby to simultaneously electrically stimulate the area of skin beneath the pad and also provide light to that area from the LEDs.

This invention relates to apparatus for artificially stimulating skin and muscles for a variety of purposes including fat mobilisation/weight reduction, skin tightening, improving the circulation and artificially exercising muscles.

A known method of fat reduction or mobilisation is to expose the skin to infra-red radiation, which may be pulsed or continuous, to thereafter break down fat, improve circulation below the surface of the skin and induce skin tightening. Such methods of fat reduction and mobilisation are surprisingly effective and it is not unusual for a reduction of up to 9 cm in the circumference of the torso to be achieved within a single treatment session lasting between 20 to 30 minutes. However, it is also well known that long term weight loss can only be achieved through regular exercise and a convenient way of achieving this is through the use of vibration-inducing exercise machines of the type described in U.S. Pat. No. 8,105,250 onto which the user can stand or sit as required to exercise the muscles of various parts of the body. A further refinement to such muscle stimulation is by the use of electrical impulses where target muscles or groups of muscles are stimulated by electrical pulses of an intensity above that required to stimulate contraction of the muscles by placing electrodes at spaced intervals over the skin to thereafter artificially stimulate muscles, such as in the manner described in GB2500642.

The various techniques described above are generally performed independently of each other, which is therefore time-consuming if two or three such techniques are required and the present invention is derived from the realisation that there is a need for an alternative approach whereby multiple functions can be performed at the same time.

According to a first aspect of the invention there is provided apparatus for applying light to an area of skin and for simultaneously electrically stimulating that area of skin, the apparatus comprising or including at least one flexible pad adapted to be placed onto or over the area of skin to be treated, the or each pad including a flexible circuit board providing, in use, electrical power to an electric muscle stimulation (EMS) electrode and an array of Light Emitting Diodes (LEDs) interspersed therebetween whereby to simultaneously electrically stimulate the area of skin beneath the pad and also provide light to that area from the LEDs.

With this arrangement multiple flexible pads may be used to treat multiple areas of skin and muscle, with each pad having an electrical connection to an operating console by which various parameters may be adjusted, such as the frequency and intensity of electrical muscle stimulation and the intensity of light emitted by the LEDs by using, for example, arrays of LEDs with different wavelengths with the operating console conveniently being self-standing such that it may be operable by a user during a treatment session. The treatment session itself may conveniently occur whilst the user is standing on an electrically operable vibration plate which may in part also stimulate muscle used during walking. Hence, with such an arrangement a variety of muscle and skin stimulation techniques which are normally done separately can instead be done concurrently.

According to a second aspect of the invention there is provided a method of treatment of an area of skin through the use of a flexible pad of the type described in the first aspect of the invention.

The invention will now be described, by way of example only, with reference to the accompanying drawings in which:

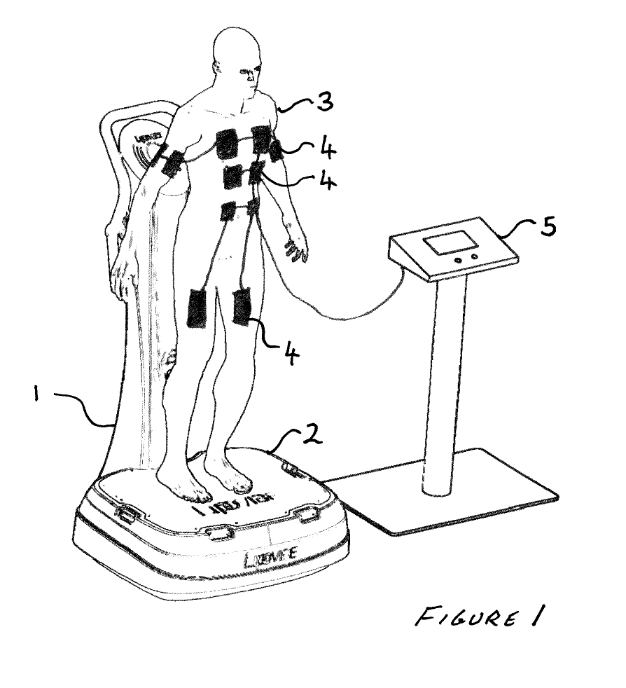

FIG. 1 is a perspective view of multi-function skin and muscle treatment pads being used on a person standing on an exercise vibration plate.

FIG. 2 is a top plan view of one of the flexible pads of FIG. 1,

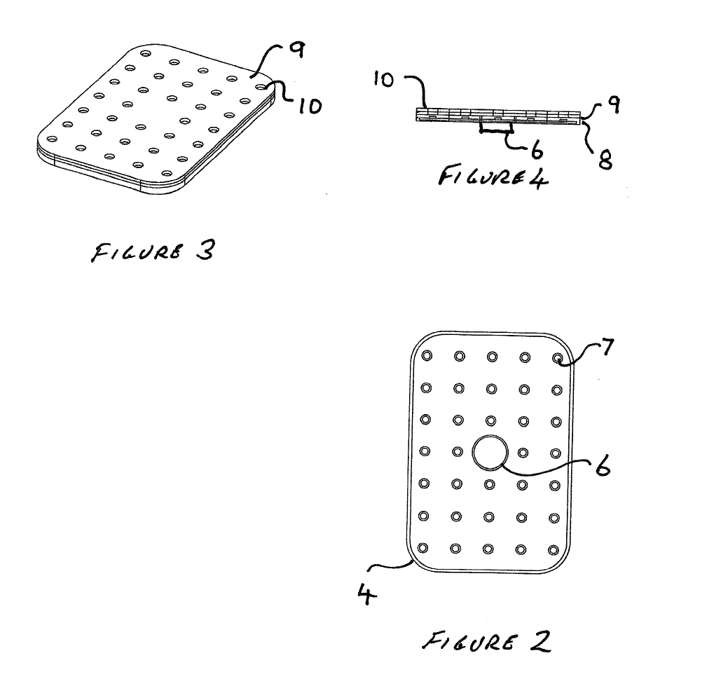

FIG. 3 is a perspective view of the opposite side of the pad of FIG. 2, and

FIG. 4 is a sectional view of FIG. 3.

Referring firstly to FIG. 1 there is shown a conventional exercise machine 1 having a vibrational plate 2 onto which a person 3 can stand and exercise e.g. leg muscles. The duration and frequency of the vibrations can be adjusted, with a typical exercise session lasting 20 minutes.

In accordance with the first aspect of the invention the person 3 is also provided with an array of flexible pads 4, typically having a silicone substrate, temporarily stuck to skin over various groups of muscles via a conductive adhesive or they may, if preferred, be secured in place by e.g. velcro straps. Each of the pads 4 is electrically connected to a floor standing console 5 by which the person 3 can make adjustments to each pad 4.

Turning to FIGS. 2 to 4, it will be seen that each pad 4 includes a central electrode 6 which supplies power to an array of LEDs 7 and a printed circuit board 8 electrically connected to an electro muscle stimulation electrode pad 9 which includes a series of apertures 10 through which light from the LEDs 7 can shine. Thus, in use, each pad 4 is able to both electrically stimulate muscles via the electrode 9 and, at the same time, shine light from the LEDs 7 to the same area of skin. As a consequence, where multiple such flexible pads 4 are used they can be used at the same time as the exercise machine 1 with the vibrational pad 2. However, whether used with or without the exercise machine 1 it has been found that the heat generated from the LEDs 7 relaxes and warms the muscles being treated via the EMS electrode 9 so that, as a consequence, the use of such light therapy and electrical muscle stimulation together has been found to be more effective and less time consuming than the use of electrical muscle stimulation or light therapy separately

1. Apparatus for applying light to an area of skin and for simultaneously electrically stimulating that area of skin, the apparatus comprising or including at least one flexible pad adapted to be placed onto or over the area of skin to be treated, the or each pad including a flexible circuit board providing, in use, electrical power to an electric muscle stimulation (EMS) electrode and an array of Light Emitting Diodes (LEDs) interspersed therebetween whereby to simultaneously electrically stimulate the area of skin beneath the pad and also provide light to that area from the LEDs.

2. A method of treatment of an area of skin through the use of a flexible pad according to claim 1.
